(19) United States
(12) Patent Application Publication
(10) Pub. No.: US 2020/0022683 A1
(43) Pub. Date: Jan. 23, 2020

(54) ULTRASOUND PROBE, ULTRASOUND DIAGNOSTIC APPARATUS, AND TRANSMISSION VOLTAGE SETTING METHOD

(71) Applicant: Konica Minolta, Inc., Tokyo (JP)

(72) Inventor: Erina KOMATSU, Tokyo (JP)

(21) Appl. No.: 16/508,586

(22) Filed: Jul. 11, 2019

(30) Foreign Application Priority Data
Jul. 20, 2018 (JP) 2018-136290

(51) Int. Cl. A61B 8/00; G01N 29/44; G01N 29/24

(52) U.S. Cl. CPC A61B 8/54; G01N 29/24; G01N 29/4436; G01N 29/4463

(57) ABSTRACT

An ultrasound probe which transmits and receives ultrasound waves, the ultrasound probe including a first storage in which probe type information of the ultrasound probe and individual property information indicating an individual property of the ultrasound probe are stored.

### BACKGROUND

### Technological Field

The present invention relates to an ultrasound probe, an ultrasound diagnostic apparatus, and a transmission voltage setting method.

### Description of the Related Art

In ultrasound diagnosis, the heart beat or the movement of an unborn baby is obtained as an ultrasound image by an easy operation of putting the ultrasound probe against the body surface or from inside the body cavity, and furthermore the patient is safe. Thus, the examination can be performed repeatedly. There is known an ultrasound diagnostic apparatus used for the ultrasound diagnosis, which has the ultrasound probe, and generates and displays the ultrasound image.

The ultrasound diagnostic apparatus includes an ultrasound probe which transmits and receives ultrasound waves, and a transmitter which outputs transmission signals (drive signals) that are electrical signals for transmitting the ultrasound waves to the ultrasound probe. A configuration for suppressing the influence of variation in parts of this transmitter is known. For example, an ultrasound diagnostic apparatus which includes an adjustment circuit that suppresses the property variation of FET (Field Effective Transistor) elements as a pair of output terminals of the transmitter is known (see Japanese Patent Application Laid Open Publication No. 2005-278666). By the adjustment circuit, it is possible to eliminate switching noise with saved power in a case where a pair of output elements have the property variation.

The ultrasound probe includes a cable which is connected to an ultrasound diagnostic apparatus main body. The entire lengths of cables may vary when they are manufactured. In the ultrasound diagnostic apparatus of Japanese Patent Application Laid Open Publication No. 2005-278666, it is possible to suppress the property variation of two output terminals which are mounted together, however, it is not possible to suppress the variation of voltages (transmission voltages) of the transmission signals based on the variation of individual properties such as the entire lengths of the cables of the ultrasound probes. When the transmission voltages vary, the image qualities of the ultrasound images vary.

As for the transmission voltage, a standard value of index for evaluating safety such as MI (Mechanical Index), TI (Thermal Index) and Ispta. 3 is determined, and adjustment of the transmission voltage is required to satisfy the standard value. Thus, when the individual properties vary between a plurality of ultrasound probes, the transmission voltages need to be uniformly lowered, and a desired image quality cannot be obtained. Especially, as the variation of individual properties is larger, the reduction amount of transmission voltage becomes larger, and the reduction amount of image quality of the ultrasound image also becomes larger.

### SUMMARY

An object of the present invention is to set an optimum transmission voltage regardless of the individual property of the ultrasound probe.

To achieve at least one of the abovementioned objects, according to a first aspect of the present invention, an ultrasound probe reflecting one aspect of the present invention is an ultrasound probe which transmits and receives ultrasound waves, the ultrasound probe including a first storage in which probe type information of the ultrasound probe and individual property information indicating an individual property of the ultrasound probe are stored.

According to a second aspect of the present invention, an ultrasound probe reflecting one aspect of the present invention is an ultrasound probe which transmits and receives ultrasound waves, the ultrasound probe including a first storage in which individual property information indicating an individual property of the ultrasound probe and reference individual property information that is a reference of the individual property information of the ultrasound probe are stored.

According to a third aspect of the present invention, an ultrasound diagnostic apparatus reflecting one aspect of the present invention is an ultrasound diagnostic apparatus, including: an ultrasound probe which transmits and receives ultrasound waves; an ultrasound diagnostic apparatus main body to which the ultrasound probe is connected; and a detector which detects individual property information indicating an individual property of the ultrasound probe, wherein the ultrasound probe includes a first storage in which probe type information of the ultrasound probe is stored, and the ultrasound diagnostic apparatus main body includes: a second storage in which reference individual property information associated with the probe type information of the ultrasound probe is stored; and a hardware processor which obtains the probe type information of the ultrasound probe from the first storage, obtains the reference individual property information corresponding to the obtained probe type information from the second storage, calculates a correction amount of a reference transmission voltage from the detected individual property information and the obtained reference individual property information, and corrects the reference transmission voltage with the calculated correction amount and sets the corrected reference transmission voltage as a transmission voltage.

According to a fourth aspect of the present invention, an ultrasound diagnostic apparatus reflecting one aspect of the present invention is an ultrasound diagnostic apparatus, including: an ultrasound probe which transmits and receives ultrasound waves; an ultrasound diagnostic apparatus main body to which the ultrasound probe is connected; and a detector which detects individual property information indicating an individual property of the ultrasound probe, wherein the ultrasound probe includes a first storage in which reference individual property information that is a reference of the individual property information of the ultrasound probe is stored, and the ultrasound diagnostic apparatus main body includes a hardware processor which obtains the reference individual property information of the ultrasound probe from the first storage, calculates a correction amount of a reference transmission voltage from the detected individual property information and the obtained reference individual property information, and corrects the reference transmission voltage with the calculated correction amount and sets the corrected reference transmission voltage as a transmission voltage.

According to a fourth aspect of the present invention, a transmission voltage setting method reflecting one aspect of the present invention is a transmission voltage setting method, including: obtaining of probe type information and individual property information of an ultrasound probe from a first storage, the ultrasound probe including the first storage in which the probe type information of the ultrasound probe and the individual property information indicating an individual property of the ultrasound probe are stored; calculating that includes obtainment of reference individual property information corresponding to the obtained probe type information from a second storage in which the reference individual property information associated with the probe type information of the ultrasound probe is stored, and that includes calculation of a correction amount of a reference transmission voltage from the individual property information and the reference individual property information which are obtained; and setting that includes correction of the reference transmission voltage with the calculated correction amount and includes setting of the corrected reference transmission voltage as a transmission voltage.

According to a fourth aspect of the present invention, a transmission voltage setting method reflecting one aspect of the present invention is a transmission voltage setting method, including: obtaining of probe type information of an ultrasound probe from a first storage, the ultrasound probe including the first storage in which the probe type information of the ultrasound probe is stored; detecting of individual property information indicating an individual property of the ultrasound probe; calculating that includes obtainment of reference individual property information corresponding to the obtained probe type information from a second storage in which the reference individual property information associated with the probe type information of the ultrasound probe is stored, and that includes calculation of a correction amount of a reference transmission voltage from the detected individual property information and the obtained reference individual property information; and setting that includes correction of the reference transmission voltage with the calculated correction amount and includes setting of the corrected reference transmission voltage as a transmission voltage.

According to a fourth aspect of the present invention, a transmission voltage setting method reflecting one aspect of the present invention is a transmission voltage setting method, including: detecting of individual property information of an ultrasound probe that includes a first storage in which reference individual property information that is a reference of the individual property information is stored, the individual property information indicating an individual property of the ultrasound probe; calculating which includes obtainment of the reference individual property information of the ultrasound probe from the first storage, and includes calculation of a correction amount of a reference transmission voltage from the detected individual property information and the obtained reference individual property information; and setting which includes correction of the reference transmission voltage with the calculated correction amount and includes setting of the corrected reference transmission voltage as a transmission voltage.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a schematic outer view of an ultrasound diagnostic apparatus in a first embodiment of the present invention;

FIG. 2 is a block diagram showing the functional configuration of the ultrasound diagnostic apparatus in the first embodiment;

FIG. 3 is a view showing the configuration of a reference transmission voltage table in the first embodiment;

FIG. 4 is a flowchart showing first transmission voltage setting processing;

FIG. 5 is a block diagram showing the functional configuration of an ultrasound diagnostic apparatus in a second embodiment;

FIG. 6 is a view showing the configuration of a reference transmission voltage table in the second embodiment; and

FIG. 7 is a flowchart showing second transmission voltage setting processing.

### DETAILED DESCRIPTION OF EMBODIMENTS

Hereinafter, one or more embodiments of the present invention will be described with reference to the drawings. However, the scope of the invention is not limited to the disclosed embodiments.

The first and second embodiments of the present invention will be described in order in detail with reference to the accompanying drawings.

### First Embodiment

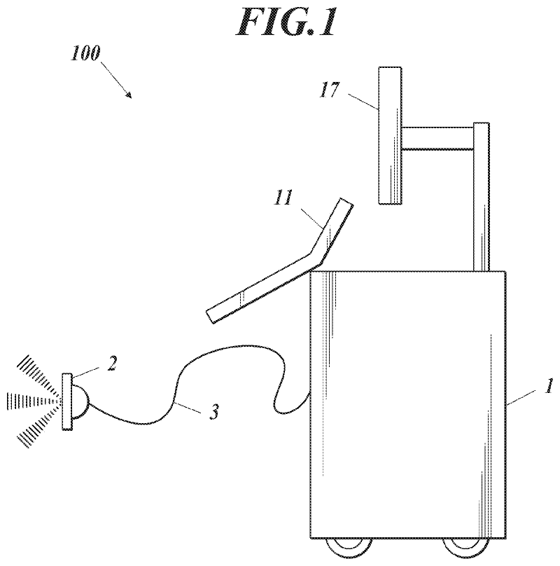

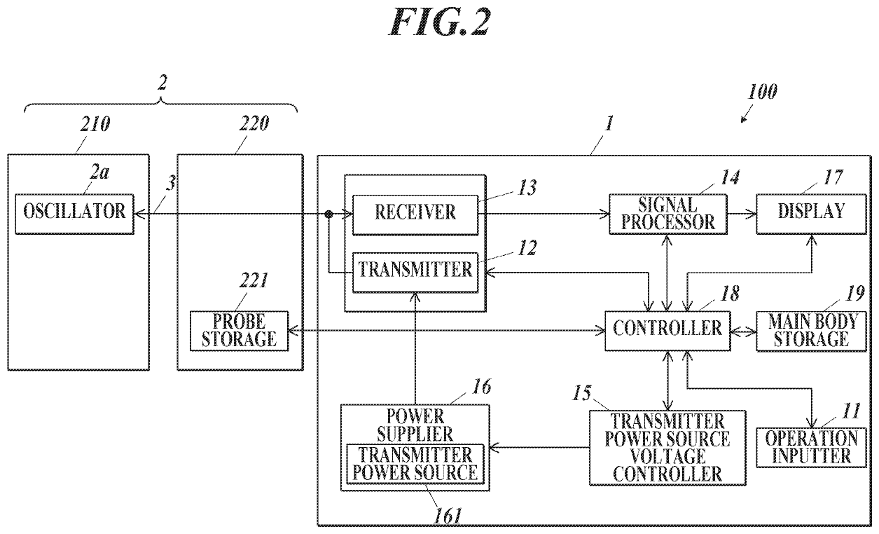

With reference to FIGS. 1 to 4, an embodiment of the present invention will be described. First, the entire apparatus configuration of an ultrasound diagnostic apparatus 100 in the embodiment will be described with reference to FIGS. 1 and 2. FIG. 1 is a schematic outer view of the ultrasound diagnostic apparatus 100 in the embodiment. FIG. 2 is a block diagram showing the functional configuration of the ultrasound diagnostic apparatus 100.

The ultrasound diagnostic apparatus 100 in the embodiment is an apparatus which is used to perform ultrasound diagnosis by an operator such as a doctor and a technician at a medical facility such as a hospital. As shown in FIGS. 1 and 2, the ultrasound diagnostic apparatus 100 includes an ultrasound probe 2 and an ultrasound diagnostic apparatus main body 1. The ultrasound probe 2 transmits ultrasound waves (transmission ultrasound waves) to a subject such as a living body of a patient not shown in the drawings, and receives reflected waves of the ultrasound waves (reflected ultrasound waves: echoes) which were reflected in the subject. The ultrasound diagnostic apparatus main body 1 is connected to the ultrasound probe 2 via a cable 3, causes the ultrasound probe 2 to transmit the transmission ultrasound waves to the subject by transmitting transmission signals (drive signals) of electrical signals to the ultrasound probe 2, and performs imaging of the internal state of the subject as an ultrasound image on the basis of the reception signals which are electrical signals generated in the ultrasound probe 2 according to the reflected ultrasound waves from inside the subject received by the ultrasound probe 2.

The ultrasound probe 2 can be attached to and detached from (connected to and released from) the ultrasound diagnostic apparatus main body 1. That is, different types of ultrasound probes 2 can be connected to the ultrasound diagnostic apparatus main body 1. The ultrasound probe 2 includes a head 210, a cable 3, and a connector 220. The head 210 is a main body of the ultrasound probe 2 and includes oscillators 2a. The cable 3 is a cable which is connected to the head 210 and the connector 220. The connector 220 is a connector which is connected to the ultrasound diagnostic apparatus main body 1.

The plurality of oscillators 2a are arranged in one-dimensional array in a direction (scanning direction (lateral direction) or vertical direction (elevation direction)), for example. In the embodiment, the ultrasound probe 2 including n (for example, n=192) oscillators 2a is used. The oscillators 2a may be arranged in the two-dimensional array. The number of the oscillators 2a may be arbitrarily designed. Though a linear electronic scanning probe is adopted for the ultrasound probe 2 in the embodiment, either of the electronic scanning type and the machine scanning type may be adopted. Any type of the linear scanning type, sector scanning type and convex scanning type may be adopted.

The connector 220 includes a probe storage 221 as a first storage which stores information regarding the ultrasound probe 2. The probe storage 221 is configured by a nonvolatile memory, and stores the probe type of the ultrasound probe 2, and the cable length showing the entire length of the cable 3. As the cable length stored in the probe storage 221, a cable length which was measured in advance when the ultrasound probe 2 was manufactured is stored, for example. The head 210 may have the probe storage 221.

As shown in FIG. 2, the ultrasound diagnostic apparatus main body 1 includes, for example, an operation inputter 11, a transmitter 12, a receiver 13, a signal processor 14, a transmitter power source voltage controller 15, a power supplier 16, a display 17, a controller 18 as an obtainer, a calculator and a setter, and a main body storage 19 as a second storage.

The operation inputter 11 includes, for example, various switches, buttons, a track ball, a mouse, a keyboard and the like for performing a command instructing start of diagnosis and for inputting data such as personal information of the subject. The operation inputter 11 outputs the operation signal to the controller 18.

The transmitter 12 is a circuit which causes the ultrasound probe 2 to generate transmission ultrasound waves by generating transmission signals which are electrical signals and supplying the transmission signals to the ultrasound probe 2, by using the power source voltage supplied from the power supplier 16, in accordance with control by the controller 18. The transmitter 12 includes, for example, a clock generation circuit, a delay circuit and a pulse generation circuit. The clock generation circuit is a circuit for generating clock signals for determining the transmission timing of the transmission signals and transmission frequency. The delay circuit is a circuit for setting the delay time for each individual path corresponding to the oscillator 2a for the transmission timing of the transmission signals and delaying the transmission of the transmission signals by the set delay time to focus the transmission beams formed of the transmission ultrasound waves. The pulse generation circuit is a circuit for generating pulse signals as transmission signals at predetermined periods.

The controller 18 inputs the transmission conditions (image mode, transmission frequency) of transmission signals and transmission voltage to the transmitter 12, and causes the transmitter 12 to generate the transmission signals corresponding to the transmission conditions and transmission voltage.

The receiver 13 is a circuit which receives reception signals which are electrical signals via the cable 3 from the ultrasound probe 2 in accordance with the control by the controller 18. The receiver 13 includes, for example, an amplifier, an A/D converting circuit and a phasing addition circuit. The amplifier is a circuit for amplifying the reception signals at a preset amplifying rate for each individual path corresponding to the oscillator 2a. The A/D converting circuit is a circuit for performing A/D conversion of the amplified reception signals. The phasing addition circuit is a circuit for aligning the time phases by providing the delay times to the reception signals, to which the A/D conversion was performed, for the respective individual paths corresponding to the respective oscillators, and adding the aligned time phases (phasing addition) to generate sound ray data.

The signal processor 14 generates B-mode image data of the B (Brightness) mode by performing envelop detection processing, log amplification and gain adjustment to the sound ray data from the receiver 13 and converting the intensity of the reception signal indicated by the sound ray data into a brightness value, in accordance with control by the controller 18. The signal processor 14 may generate ultrasound image data of image modes other than the B-mode such as a color Doppler mode.

In accordance with control by the controller 18, the signal processor 14 stores the generated B-mode image data in an image memory (not shown in the drawings) inside by the unit of frame. The image data by the unit of frame may be referred to as ultrasound image data or frame image data. The signal processor 14 reads out the ultrasound image data stored in the image memory as needed. The image memory is configured by including a semiconductor memory such as a DRAM (Dynamic Random Access Memory), for example.

In accordance with control by the controller 18, the signal processor 14 performs a coordinate conversion or the like to the read frame image data to convert the data into an image signal and outputs the image signal to the display 17.

The transmitter power source voltage controller 15 is a circuit which performs control of power source voltage that is output by the power supplier 16, in accordance with the control by the controller 18. The power supplier 16 includes a transmitter power source 161. In accordance with the control by the transmitter power source voltage controller 15, the power supplier 16 causes the transmitter power source 161 to generate the power source voltage for the transmitter 12 on the basis of the power supply from a commercial power source or a battery which is not shown in the drawings. The transmitter power source 161 can be configured by including a regulator, for example.

As the display 17, there can be applied a display device such as an LCD (Liquid Crystal Display), a CRT (Cathode-Ray Tube) display, an organic EL (Electronic Luminescence) display, an inorganic EL display and plasma display. The display 17 performs display of an image on the display screen in accordance with the image signal output from the signal processor 14.

The controller 18 includes, for example, a CPU (Central Processing Unit), a ROM (Read Only Memory) and a RAM (Random Access Memory), reads out various processing programs such as a system program or the like stored in the ROM and loads them onto the RAM, and controls the sections of the ultrasound diagnostic apparatus 100 in accordance with the loaded programs. The ROM is formed of a nonvolatile memory or the like such as a semiconductor, and stores a system program corresponding to the ultrasound diagnostic apparatus 100, various processing programs executable on the system program, and various types of data such as a gamma table. The ROM especially stores an ultrasound image display program for executing ultrasound image display processing of generating and displaying the ultrasound image data, and a first transmission voltage setting program for executing first transmission voltage setting processing to be described later. These programs are stored in a form of computer readable program code, and the CPU sequentially executes the operation according to the program code. The RAM forms a working area for temporarily storing various programs executed by the CPU and data according to these programs.

The main body storage 19 is a storage which is configured by a nonvolatile memory and stores information such as a reference transmission voltage table 40 to be described later and reference transmission voltages corresponding to the various transmission conditions.

A part or all of the functions of the respective functional sections included in the ultrasound diagnostic apparatus 100 can be achieved as a hardware circuit such as an integrated circuit. The integrated circuit is an LSI (Large Scale Integration), for example, and the LSI is also called as an IC (Integrated Circuit), a system LSI, a super LSI, or an ultra LSI, according to the difference in integration degree. The method of making an integrated circuit is not limited to LSI, and the integrated circuit may be achieved by a dedicated circuit or a general-purpose processor. A reconfigurable processor which can reconfigure the connection or setting of the circuit cell inside the LSI and a FPGA (Field Programmable Gate Array) may be used. A part or all of the functions of the respective functional blocks may be achieved by software. In this case, this software is stored in one or more storage medium such as a ROM, an optical disk, a hard click or the like, and the software is executed by an arithmetic processor.

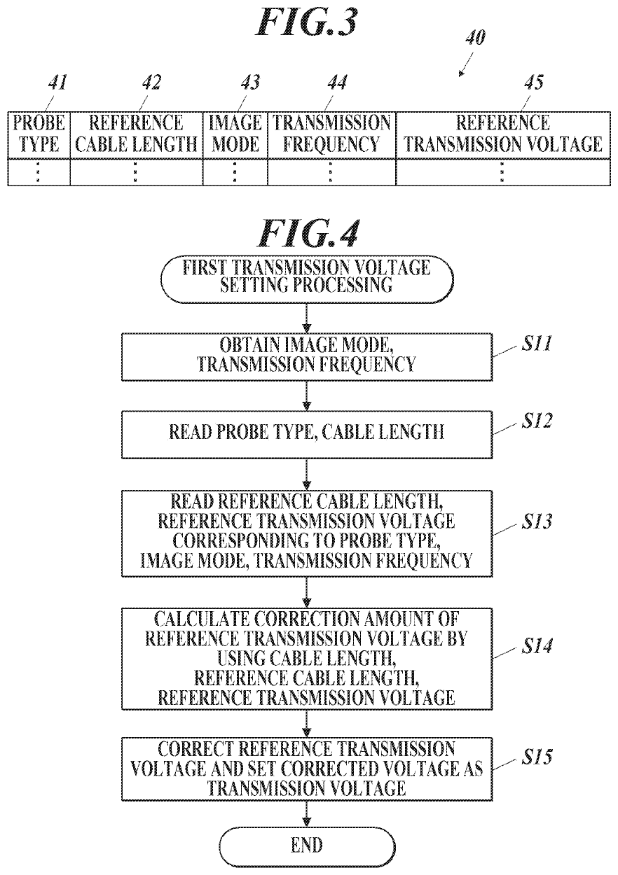

Next, with reference to FIG. 3, information stored in the ultrasound diagnostic apparatus 100 will be described. FIG. 3 is a view showing the configuration of a reference transmission voltage table 40.

The probe storage 221 which is provided in the connector 220 of the ultrasound probe 2 stores the probe type and the cable length of the cable 3 of the ultrasound probe as individual property information indicating the individual property of the ultrasound probe. The probe type is identification information of the type of the ultrasound probe 2, and the information includes information on the scanning type such as convex. The cable length is the entire length of the cable 3, and cable lengths for a same probe type are different in some cases due to the variation when the cables 3 were manufactured.

The main body storage 19 which is provided in the ultrasound diagnostic apparatus main body 1 stores a reference transmission voltage table 40 shown in FIG. 3. The reference transmission voltage table 40 has fields of the probe type 41, the reference cable length 42 as reference individual property information indicating the reference of the individual property of the probe, the image mode 43 and the transmission frequency 44 as the transmission conditions, and the reference transmission voltage 45. The probe type 41 is the probe type of at least one type of ultrasound probe which can be connected to the ultrasound diagnostic apparatus main body 1. The reference cable length 42 is the entire length which is a reference of the cable 3 of the ultrasound probe corresponding to the probe type 41. As the reference cable length 42, for example, the average value of the entire lengths of cables 3 of a plurality of ultrasound probes which are the same probe type 41, or the design value of the entire length of the cable 3 of the ultrasound type 41 is set.

The image mode 43 is a diagnosis mode which is used for obtaining and diagnosing the ultrasound image. The image mode 43 is B mode, color Doppler mode, or the like. In the embodiment, the image mode 43 can be set in the ultrasound image display processing. Some of the image modes combine ultrasound image data of two types of image modes and display the combined data. For example, when the image mode to be displayed is set to a color Doppler mode, the color blood flow image data of the color Doppler mode and the b mode image data are combined and displayed. In this case, the image mode 43 represents the image modes of the ultrasound images before the combination.

The transmission frequency 44 is a frequency of the transmission signal which can be set in the ultrasound image display processing. The transmission frequency 44 is represented by the numerical value and/or the waveform of the frequency of the transmission signal. The image mode 43 and the transmission frequency 44 are the transmission conditions other than the transmission voltage for generating and outputting the transmission signals, and the transmission conditions are not limited to them.

The reference transmission voltage 45 is a transmission voltage which is a reference corresponding to the probe type 41, the reference cable length 42, the image mode 43, the transmission frequency 44, and the like. The reference transmission voltage 45 is a voltage which corresponds to the reference cable length 42 and satisfies a standard value satisfying the safety index such as MI, TI and Ispta. 3, and which is a highest possible transmission voltage.

Next, with reference to FIG. 4, the operation of the ultrasound diagnostic apparatus 100 will be described. FIG. 4 is a flowchart showing first transmission voltage setting processing.

The ultrasound diagnostic apparatus 100 executes as needed ultrasound image display processing of generating the transmission signal and inputting the transmission signal to the ultrasound probe 2 according to the transmission conditions such as the image mode and the transmission frequency and the transmission voltage, causing the ultrasound probe 2 to transmit and receive the ultrasound waves, and generating the ultrasound image data and displaying the ultrasound image data on the display 17. The controller 18 executes the first transmission voltage setting processing according to the first transmission voltage setting program which is stored in the ROM, in response to a trigger of start to execute the ultrasound image display processing, change of the connected ultrasound probe 2 in the ultrasound image display processing which is currently executed, or input to change the transmission condition performed from the operator via the operation inputter 11, in the ultrasound diagnostic apparatus 100.

As shown in FIG. 4, first, the controller 18 obtains the image mode and the transmission frequency which are currently set (or which were changed immediately before the processing) (step S11). The controller 18 reads out the probe type and the cable length of the ultrasound probe 2 which is currently connected from the probe storage 221 (step S12).

The controller 18 then refers to the reference transmission voltage table 40 stored in the main body storage 19, and reads out the reference cable length 42 and the reference transmission voltage 45 corresponding to the probe type 41 which was read out in step S12, and corresponding to the image mode 43 and the transmission frequency 44 which were read out in step S11 (step S13). The controller 18 calculates the correction amount of the reference transmission voltage 45 which was read out in step S13 by using the cable length of the cable 3 which was read out in step S12 and the reference cable length which was read out in step S13 (step S14). If the cable length is shorter than the reference cable length, the impedance of the cable 3 is also lower. Thus, in step S14, for example, the correction amount of the reference transmission voltage 45 is calculated from the ratio of the cable length to the reference cable length 42.

The controller 18 corrects the reference transmission voltage 45 which was read out in step S13 with the correction amount which was calculated in step S14, and sets the corrected reference transmission voltage as the transmission voltage for the transmitter 12 (step S15), and ends the first transmission voltage setting processing.

In the first transmission voltage setting processing, in a case where the image mode of the ultrasound image display processing requires combination of two types of ultrasound image data, steps S11 to S15 are executed for the two image modes before the combination.

As described above, according to the embodiment, the ultrasound probe 2 which transmits and receives the ultrasound waves to and from the subject includes the probe storage 221 which stores the probe type of the ultrasound probe 2 and the individual property information indicating the individual property of the ultrasound probe 2. Thus, by using the individual property information of the ultrasound probe 2 and correcting the reference transmission voltage, it is possible to set the optimum transmission voltage and improve the image quality of the ultrasound image regardless of the individual property of the ultrasound probe 2.

The ultrasound diagnostic apparatus 100 includes an ultrasound probe 2 and an ultrasound diagnostic apparatus main body 1 which is connected to the ultrasound probe 2. In the ultrasound diagnostic apparatus main body 1, the main body storage 19 stores the reference individual property information associated with the probe type of the ultrasound probe, the probe type and the individual property information of the ultrasound probe 2 are obtained from the probe storage 221, the reference individual property information corresponding to the obtained probe type information is obtained from the main body storage 19, the correction amount of the reference transmission voltage is calculated from the obtained individual property information and the reference individual property information, and the reference transmission voltage is corrected with the calculated correction amount and the corrected voltage is set as the transmission voltage. Thus, the optimum transmission voltage can be set regardless of the individual property of the ultrasound probe 2.

The main body storage 19 stores the reference individual property information which is associated with the probe type of the ultrasound probe and the transmission conditions (image mode, transmission frequency). The ultrasound diagnostic apparatus main body 1 obtains the probe type and the individual property information of the ultrasound probe 2 from the probe storage 221, obtains the image mode and the transmission frequency which are set, and obtains the reference individual property information corresponding to the obtained probe type and the transmission conditions from the main body storage 19. Thus, it is possible to set a more optimum transmission voltage matching the transmission conditions, regardless of the individual property of the ultrasound probe 2.

The ultrasound probe 2 includes a cable 3 for connection to the ultrasound diagnostic apparatus main body 1. The individual property information is the cable length of the cable 3. Thus, it is possible to set the optimum transmission voltage regardless of the variation of the cable length of the cable 3 of the ultrasound probe 2.

In the embodiment, the cable length of the cable 3 is used as the individual property information of the ultrasound probe causing the variation of the transmission voltage, and the correction amount of the transmission voltage is calculated to correct the transmission voltage. However, the present invention is not limited to this. Other information such as impedance of the ultrasound probe 2 and the end voltage applied to both ends of the oscillator 2a may be used as the individual property information. In the configuration of using the impedance of the ultrasound probe 2 including the cable 3 as the individual property information, the probe type and the impedance of the ultrasound probe 2 which was measured in advance are stored in the probe storage 221, the reference impedance which is a reference of the impedance of the ultrasound probe is stored in the reference transmission voltage table 40 instead of the reference cable length 42. For example, considering that the impedance is higher as the cable length of the cable 3 is longer, the correction amount of the reference transmission voltage is calculated from the impedance of the ultrasound probe 2 and the reference impedance. Thus, the optimum transmission voltage can be set regardless of the variation of the impedance of the ultrasound probe 2.

In the configuration of using the end voltage as the individual property information, the probe storage 221 stores the probe type, the end voltage which was measured in advance for each image mode and transmission frequency, and the reference end voltage which is a reference of the end voltage is stored instead of the reference cable length 42 in the reference transmission voltage table 40. The correction amount of the transmission voltage is calculated from the reference end voltage and the end voltage corresponding to the image mode and the transmission frequency set in the ultrasound image display processing. Thus, it is possible to set the optimum transmission voltage regardless of the variation of the end voltage based on the cable length or the like of the ultrasound probe 2.

In the embodiment, the reference transmission voltage table 40 is stored in the main body storage 19. However, the present invention is not limited to this. The reference transmission voltage table 40 may be stored in the probe storage 221. In this configuration, information regarding only the ultrasound probe 2 which has the probe storage 221 to store the reference transmission voltage table 40 is stored. The probe type 41 may not be necessary. For example, the ultrasound probe 2 includes the probe storage 221 which stores individual property information (cable length of cable 3, end voltage of the oscillator 2a or impedance of ultrasound probe 2) indicating the individual property of the ultrasound probe 2, the reference individual property information which is a reference of the individual property information of the ultrasound probe 2, and the reference transmission voltage which is a reference of the transmission voltage of the ultrasound probe 2. Thus, it is possible to set the optimum transmission voltage regardless of the individual property of the ultrasound probe 2, by using the individual property information and the reference individual property information of the ultrasound probe 2 and correcting the reference transmission voltage stored in the ultrasound probe 2.

### Second Embodiment

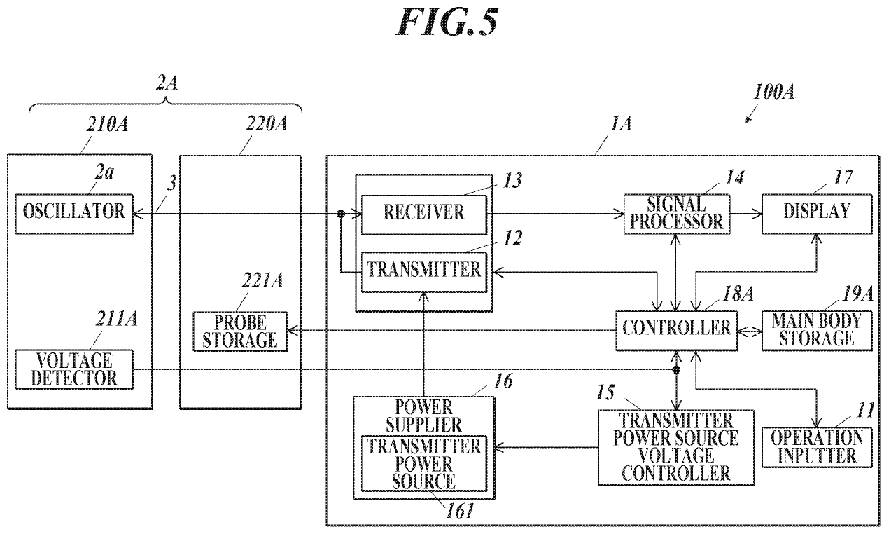

With reference to FIGS. 5 to 7, a second embodiment of the present invention will be described. First, with reference to FIG. 5, the apparatus configuration of the embodiment will be described. However, as for the same parts as those of the ultrasound diagnostic apparatus 100 of the first embodiment in the apparatus configuration of the second embodiment, the explanation is omitted by providing same reference numerals. FIG. 5 is a block diagram showing the functional configuration of an ultrasound diagnostic apparatus 100A in the second embodiment.

As shown in FIG. 5, the ultrasound diagnostic apparatus 100A in the embodiment includes an ultrasound probe 2A and an ultrasound diagnostic apparatus main body 1A. The ultrasound probe 2A includes a head 210A, a cable 3, and a connector 220A.

The head 210A includes an oscillator 2a and a voltage detector 211A. The voltage detector 211A is configured by including a same detection circuit as the impedance analyzer, and detects the end voltage applied to the oscillator 2a, for example.

The connector 220A includes the probe storage 221A. The probe storage 221A is similar to the probe storage 221 in the first embodiment, but stores the probe type of the ultrasound probe 2A.

The ultrasound diagnostic apparatus main body 1A includes, for example, an operation inputter 11, a transmitter 12, a receiver 13, a signal processor 14, a transmitter power source voltage controller 15, a power supplier 16, a display 17, a controller 18A, and a main body storage 19A.

The controller 18A is similar to the controller 18 in the first embodiment, but stores an ultrasound image display program, a second transmission voltage setting program for executing second transmission voltage setting processing to be described later, and the like in the ROM inside the controller 18A.

The main body storage 19A is similar to the main body storage 19 in the first embodiment, but a reference transmission voltage table 50 to be described later is stored.

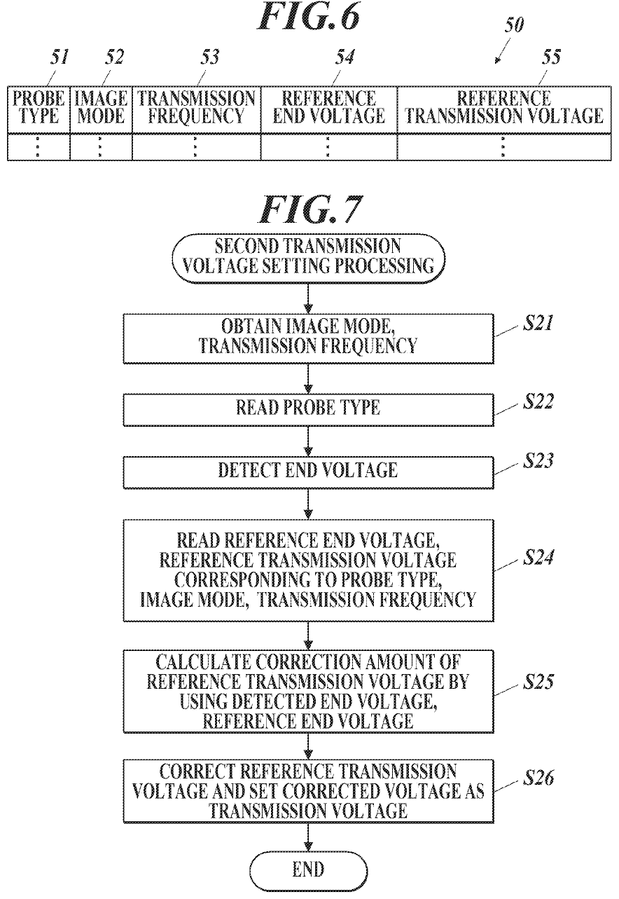

Next, information stored in the ultrasound diagnostic apparatus 100A will be described with reference to FIG. 6. FIG. 6 is a view showing the configuration of the reference transmission voltage table 50.

The probe type as identification information of the type of the ultrasound probe 2A is stored in the probe storage 221A which is included in the connector 220A of the ultrasound probe 2A.

The reference transmission voltage table 50 is stored in the main body storage 19A included in the ultrasound diagnostic apparatus main body 1A. The reference transmission voltage table 50 has fields of the probe type 51, the image mode 52 and the transmission frequency 53 as the transmission conditions, the reference end voltage 54 as the individual property information, and the reference transmission voltage 55. The probe type 51 is the probe type of at least one type of the ultrasound probe which can be connected to the ultrasound diagnostic apparatus main body 1A. The image mode 52 is an image mode such as a B-mode and a color Doppler mode which can be set in the ultrasound image display processing. In a case where the ultrasound image data of two types of image modes is combined, the image mode 52 is the image modes before combination. The transmission frequency 53 is the frequency of the transmission signal which can be set in the ultrasound image display processing.

The reference end voltage 54 is the end voltage which is a reference corresponding to the probe type 51, the image mode 52 and the transmission frequency 53, and applied to both ends of the oscillator 2a of the ultrasound probe 2A. The actual end voltage applied to both ends of the oscillator 2a is changed due to the variation of cable length of the cable 3 or impedance of ultrasound probe 2A, time degradation and the like even for the same probe type 51, image mode 52, and transmission frequency 53. The reference end voltage 54 is the end voltage which is corresponding to the reference cable length of the cable 3 or the reference impedance of the ultrasound probe 2A and which is a reference when the time degradation is not generated. The reference transmission voltage 55 is a transmission voltage which is corresponding to the probe type 51, the image mode 52 and the transmission frequency 53, and corresponding to the reference cable length of the cable 3 or the reference impedance of the ultrasound probe 2A, and which is a reference when the time degradation is not generated. The reference transmission voltage 55 is a voltage which satisfies the standard value satisfying the safety index such as MI, TI, and Ispta. 3, and is a highest possible transmission voltage.

Next, with reference to FIG. 7, the operation of the ultrasound diagnostic apparatus 100A will be described. FIG. 7 is a flowchart showing second transmission voltage setting processing.

Similarly to the first embodiment, the ultrasound diagnostic apparatus 100A executes as needed the ultrasound image display processing. The controller 18A executes the second transmission voltage setting processing according to the second transmission voltage setting program which is stored in the ROM, in response to a trigger of start to execute the ultrasound image display processing, change of the connected ultrasound probe 2 in the ultrasound image display processing which is currently executed, or input to change the transmission condition performed from the operator via the operation inputter 11, in the ultrasound diagnostic apparatus 100A.

As shown in FIG. 7, first, step S21 is similar to step S11 of first transmission voltage setting processing in FIG. 4. The controller 18A reads out the probe type of the ultrasound probe 2A which is currently connected from the probe storage 221 (step S22).

The controller 18A detects the end voltage applied to both ends of the oscillator 2a by the voltage detector 211A (step S23). The controller 18A refers to the reference transmission voltage table 50 stored in the main body storage 19, and reads out the reference end voltage 54 corresponding to the probe type 51 which was read out in step S22, and the image mode 52 and the transmission frequency 53 which were obtained in step S21 (step S24).

The controller 18A calculates the correction amount of the reference transmission voltage by using the end voltage which was detected in step S23 and the reference end voltage which was read in step S24 (step S25). Even if the transmission voltage is set to the value corresponding to the reference end voltage, there is a concern that the end voltage cannot be the reference end voltage due to the variation of the cable length of the cable 3 or the impedance of the ultrasound probe 2A, the time degradation, and the like. Thus, in step S25, the correction amount of the reference transmission voltage for setting the end voltage applied to both ends of the oscillator to the reference end voltage is calculated from the ratio of the end voltage to the reference end voltage, for example.

The controller 18A corrects the reference transmission voltage, which was read out in step S24, with the correction amount which was calculated in step S25, sets the corrected reference transmission voltage as the transmission voltage for the transmitter 12 (step S26), and ends the second transmission voltage setting processing. In the second transmission voltage setting processing, in a case where the image mode requires combination of two types of ultrasound image data, steps S21 to S26 are executed for the two image modes before the combination.

As described above, according to the embodiment, the ultrasound diagnostic apparatus 100A includes: an ultrasound probe 2A which transmits and receives ultrasound waves to and from the subject; and an ultrasound diagnostic apparatus main body 1A which is connected to the ultrasound probe 2A. The ultrasound probe 2A includes: a probe storage 221A which stores the probe type of the ultrasound probe 2A; and a voltage detector 211A which detects individual property information indicating the individual property of the ultrasound probe 2A. The ultrasound diagnostic apparatus main body 1A includes a main body storage 19A which stores the reference individual property information associated with the probe type of the ultrasound probe. The probe type of the ultrasound probe 2A is obtained from the probe storage 221A, the reference individual property information corresponding to the obtained probe type is obtained from the main body storage 19A, the correction amount of the reference transmission voltage is calculated from the detected individual property information and the obtained reference individual property information, and the reference transmission voltage is corrected with the calculated correction amount and the corrected voltage is set as the transmission voltage. Thus, by detecting the individual property information of the ultrasound probe 2A and feeding back the detected information, the optimum transmission voltage can be set and the image quality of the ultrasound image can be improved regardless of the individual property of the ultrasound probe 2A.

The individual property information is the end voltage applied to the oscillator 2a. Thus, it is possible to set the optimum transmission voltage regardless of the variation of the end voltage of the ultrasound probe 2A at the time of manufacturing or the variation of the end voltage based on the time degradation, and the like.

In the embodiment, the correction amount of the transmission voltage is calculated to perform correction of the transmission voltage, by using the end voltage applied to both ends of the oscillator 2a as the detection target of the ultrasound probe 2A. However, the present invention is not limited to this. As the detection target of the ultrasound probe 2A, the impedance of the ultrasound probe 2A including the cable 3 may be used. In the configuration of detecting the impedance of the ultrasound probe 2A, for example, the ultrasound diagnostic apparatus main body 1A or the ultrasound probe 2A includes an impedance detector similar to the detection circuit of the impedance analyzer, and the impedance of the ultrasound probe 2A is detected by the impedance detector. The correction amount of the reference transmission voltage is calculated from the detected impedance of the ultrasound probe 2A and the reference impedance of the ultrasound probe 2A. Thus, the optimum transmission voltage can be set regardless of the variation of the impedance of the ultrasound probe 2A at the time of manufacturing and the variation of the impedance of the ultrasound probe 2A based on the time degradation and the like.

In the embodiment, the reference transmission voltage table 50 has the reference end voltage 54. However, the present invention is not limited to this. The reference transmission voltage table 50 may not have the reference end voltage 54 by assuming that the end voltage is equal to the transmission voltage. In this configuration, in step S25 of the second transmission voltage setting processing in FIG. 7, the correction amount of the reference transmission voltage is calculated from the end voltage which was detected in step S23, and the reference transmission voltage which was read out in step S24. For example, the correction amount of the reference transmission voltage is calculated from the ratio of the end voltage to the reference transmission voltage.

In the embodiment, the reference transmission voltage table 50 is stored in the main body storage 19A. The present invention is not limited to this. The reference transmission voltage table 50 may be stored in the probe storage 221A. In this configuration, information regarding only the ultrasound probe 2A which has the probe storage 221A to store the reference transmission voltage table 50 is stored. The probe type 51 may not be necessary. For example, the ultrasound probe 2A of the ultrasound diagnostic apparatus 100A includes: the voltage detector 211A which detects the individual property information (end voltage of oscillator 2a) indicating the individual property of the ultrasound probe 2A; and the probe storage 221A which stores, as the reference transmission voltage table 50, the reference individual property information that is a reference of the individual property information of the ultrasound probe 2A, and the reference transmission voltage. The ultrasound diagnostic apparatus main body 1A obtains the reference individual property information of the ultrasound probe 2A from the probe storage 221A, calculates the correction amount of the reference transmission voltage from the detected individual property information and the obtained reference individual property information, corrects the reference transmission voltage 55 with the calculated correction amount and sets the corrected voltage as the transmission voltage. The individual property information may be the impedance of the ultrasound probe 2A. Thus, it is possible to set the optimum transmission voltage regardless of the variation of the individual property information of the ultrasound probe 2A at the time of manufacturing and the variation of the individual property information based on the time degradation and the like.

The description of the above embodiments are examples of the preferred ultrasound probe and the ultrasound diagnostic apparatus according to the present invention, and the present invention is not limited to this. For example, all or a part of the above embodiments may be combined as needed.

As for the detailed configurations and detailed operations of the sections forming the ultrasound diagnostic apparatuses 100, 100A in the embodiments, modifications can be made as needed within the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese patent Application No. 2018-136290, filed on 20, Jul. 2018, including description, claims, drawings and abstract is incorporated herein by reference in its entirety.

1. An ultrasound probe which transmits and receives ultrasound waves, the ultrasound probe comprising a first storage in which probe type information of the ultrasound probe and individual property information indicating an individual property of the ultrasound probe are stored.

2. The ultrasound probe according to claim 1, comprising a cable for connection to an ultrasound diagnostic apparatus main body which causes the ultrasound probe to transmit and receive the ultrasound waves, wherein the individual property information is a length of the cable.

3. The ultrasound probe according to claim 1, wherein the individual property information is an impedance of the ultrasound probe.

4. The ultrasound probe according to claim 1, comprising an oscillator which transmits and receives the ultrasound waves, wherein the individual property information is an end voltage applied to the oscillator.

5. An ultrasound probe which transmits and receives ultrasound waves, the ultrasound probe comprising a first storage in which individual property information indicating an individual property of the ultrasound probe and reference individual property information that is a reference of the individual property information of the ultrasound probe are stored.

6. An ultrasound diagnostic apparatus, comprising:
the ultrasound probe according to claim 1; and
an ultrasound diagnostic apparatus main body to which the ultrasound probe is connected, wherein
the ultrasound diagnostic apparatus main body includes:
a second storage in which reference individual property information associated with the probe type information of the ultrasound probe is stored; and
a hardware processor which obtains the probe type information and the individual property information of the ultrasound probe from the first storage, obtains the reference individual property information corresponding to the obtained probe type information from the second storage, calculates a correction amount of a reference transmission voltage from the individual property information and the reference individual property information that are obtained, and corrects the reference transmission voltage with the calculated correction amount and sets the corrected reference transmission voltage as a transmission voltage.

7. An ultrasound diagnostic apparatus, comprising:
an ultrasound probe which transmits and receives ultrasound waves;
an ultrasound diagnostic apparatus main body to which the ultrasound probe is connected; and
a detector which detects individual property information indicating an individual property of the ultrasound probe, wherein
the ultrasound probe includes a first storage in which probe type information of the ultrasound probe is stored, and
the ultrasound diagnostic apparatus main body includes:
a second storage in which reference individual property information associated with the probe type information of the ultrasound probe is stored; and
a hardware processor which obtains the probe type information of the ultrasound probe from the first storage, obtains the reference individual property information corresponding to the obtained probe type information from the second storage, calculates a correction amount of a reference transmission voltage from the detected individual property information and the obtained reference individual property information, and corrects the reference transmission voltage with the calculated correction amount and sets the corrected reference transmission voltage as a transmission voltage.

8. The ultrasound diagnostic apparatus according to claim 7, wherein the individual property information is an impedance of the ultrasound probe.

9. The ultrasound diagnostic apparatus according to claim 7, wherein
the ultrasound probe includes an oscillator which transmits and receives ultrasound waves, and
the individual property information is an end voltage applied to the oscillator.

10. The ultrasound diagnostic apparatus according to claim 6, wherein
the reference individual property information associated with the probe type information of the ultrasound probe and a transmission condition is stored in the second storage, and
the hardware processor obtains the transmission condition which is set, and obtains, from the second storage, the reference individual property information corresponding to the probe type information and the transmission condition which are obtained.

11. The ultrasound diagnostic apparatus according to claim 10, wherein a transmission frequency is stored as the transmission condition in the second storage.

12. An ultrasound diagnostic apparatus, comprising:
an ultrasound probe which transmits and receives ultrasound waves;
an ultrasound diagnostic apparatus main body to which the ultrasound probe is connected; and
a detector which detects individual property information indicating an individual property of the ultrasound probe, wherein
the ultrasound probe includes a first storage in which reference individual property information that is a reference of the individual property information of the ultrasound probe is stored, and
the ultrasound diagnostic apparatus main body includes a hardware processor which obtains the reference individual property information of the ultrasound probe from the first storage, calculates a correction amount of a reference transmission voltage from the detected individual property information and the obtained reference individual property information, and corrects the reference transmission voltage with the calculated correction amount and sets the corrected reference transmission voltage as a transmission voltage.

13. (canceled)

14. (canceled)

15. (canceled)
